(19) United States
(12) Patent Application Publication
(10) Pub. No.: US 2025/0283158 A1
(43) Pub. Date: Sep. 11, 2025

(54) BEACON-MEDIATED EXPONENTIAL AMPLIFICATION REACTION (BEAR) USING A SINGLE ENZYME AND PRIMER

(71) Applicant: The Governors of the University of Alberta, Edmonton (CA)

(72) Inventors: Xiaochun Chris Le, Edmonton (CA); Hongquan Zhang, Ottawa (CA); Ashley Meagan Newbigging, Edmonton (CA)

(21) Appl. No.: 19/217,836

(22) Filed: May 23, 2025

Related U.S. Application Data
(63) Continuation of application No. 16/883,734, filed on May 26, 2020, now abandoned.
(60) Provisional application No. 62/852,390, filed on May 24, 2019.

(51) Int. Cl. C12Q 1/6844

(52) U.S. Cl. CPC C12Q 1/6844

(57) ABSTRACT

There is described Beacon-mediated Exponential Amplification Reaction (BEAR) Using a Single Enzyme and Primer.

### SEQUENCE LISTING

The instant application contains a Sequence Listing which has been submitted electronically in XML format and is hereby incorporated by reference in its entirety. Said XML copy, created May 23, 2025, is named 51012-033003_Sequence_Listing_5_23_25.xml and is 15,277 bytes in size.

### FIELD

The present disclosure relates generally to Beacon-mediated Exponential Amplification Reaction (BEAR) Using a Single Enzyme and Primer.

### BACKGROUND

Exponential and isothermal amplification of nucleic acid targets is highly desirable because it retains the extraordinary amplification power of the polymerase chain reaction (PCR) while obviating the need for thermal cycling. Each cycle of PCR requires changes of temperatures between lower temperatures for annealing (e.g., 60° C.) and extension (e.g., 70° C.), and a higher temperature for denaturation (e.g., 95° C.) of double stranded DNA (dsDNA) to single stranded DNA (ssDNA). Eliminating thermal cycling would allow the use of simpler devices, and facilitate the potential for point-of-care testing particularly in resource limited settings. Several isothermal and exponential amplification techniques have been developed, such as exponential strand displacement amplification (E-SDA), exponential rolling circle amplification (E-RCA), exponential amplification reaction (EXPAR), nucleic acid sequence based amplification (NASBA), helicase-dependent amplification (HDA), and recombinase polymerase amplification (RPA). Although these techniques do not need to use a high temperature for denaturation, they require enzymes in addition to the polymerase, to help generate ssDNA from dsDNA. EXPAR[5a] and SDA use the cooperation of an endonuclease and a polymerase to generate ssDNA. HDA and RPA use helicase and recombinase, respectively, to convert dsDNA to ssDNA. One exception is loop-mediated isothermal amplification (LAMP), which uses only a polymerase. However, LAMP requires four to six primers, which complicates the design.

Limiting the number of required enzymes and primers would simplify technical procedures and reduce the restrictions on reagent storage requirements, both of which can extend the applicability of amplification techniques for use in point-of-care testing. Despite many recent advances in isothermal methods with exponential amplification, there is no technique that uses a single enzyme and a single primer for isothermal and exponential amplification.

### SUMMARY

In one aspect there is provide a Beacon-mediated Exponential Amplification Reaction (BEAR) system, comprising;
a polynucleotide beacon, comprising:
   a polynucleotide hairpin (HP), comprising: a first overhang region comprising a fluorophore quencher conjugated to the 5′end of said first overhang region, a first stem region positioned 3′ to the first overhang region, a loop region positioned 3′ to the first stem region, a second stem region positioned 3′ to the loop region, such second stem region complementary to the first stem region, and a second overhang region positioned 3′ to the second stem region;
   a fluorophore-conjugated polynucleotide (FS), comprising a fluorophore moiety conjugated to the 3′end of said polynucleotide, said fluorophore-conjugated polynucleotide is adapted to reversibly bind to said first overhang region of said polynucleotide hairpin (HP), and said first stem region and loop region of said polynucleotide hairpin (HP),
a polynucleotide primer configured to reversibly bind to a primer binding region that overlaps with said second stem region and said second overhang region of said polynucleotide hairpin (HP) when a target polynucleotide in a sample reversibly binds to said loop region of said polynucleotide hairpin (HP), or when an unbound fluorophore-conjugated polynucleotide (FS) reversibly binds to said first stem region and said loop region of said polynucleotide hairpin (HP); and
a polypeptide with DNA polymerase activity.

In one example, wherein each said first overhang region, said first stem region, said loop region, said second stem region, or said primer binding region comprises one or more nucleotide domains.

In one example, wherein the concentration of the polynucleotide hairpin (HP) is in excess of the concentration of the fluorophore-conjugated polynucleotide (FS).

In one example, wherein said first stem region or second stem region comprises or consists of 14 nucleotides.

In one example, wherein said first stem region or second stem region comprises or consists of about 57% content of G and C nucleotide.

In one example, wherein the polynucleotide primer comprises or consist of 8 to 11 nucleotides.

In one example, wherein said fluorophore moiety is fluorescein amidite (FAM™).

In one example, wherein said quencher is IOWA BLACK™ FQ.

In one aspect there is provided a method of detecting a target polynucleotide in a sample, comprising: contacting a sample with a Beacon-mediated Exponential Amplification Reaction (BEAR) system, said system comprising;
a polynucleotide beacon, comprising:
   a polynucleotide hairpin (HP), comprising: a first overhang region comprising a fluorophore quencher conjugated to the 5′end of said first overhang region, a first stem region positioned 3′ to the first overhang region, a loop region positioned 3′ to the first stem region, a second stem region positioned 3′ to the loop region, such second stem region complementary to the first stem region, and a second overhang region positioned 3′ to the second stem region;
   a fluorophore-conjugated polynucleotide (FS), comprising a fluorophore moiety conjugated to the 3′end of said polynucleotide, said fluorophore-conjugated polynucleotide is adapted to reversibly bind to said first overhang region of said polynucleotide hairpin (HP), and said first stem region and loop region of said polynucleotide hairpin (HP),
a polynucleotide primer configured to reversibly bind to a primer binding region that overlaps with said second stem region and said second overhang region of said polynucleotide hairpin (HP) when a target polynucleotide in a sample reversibly binds to said loop region of said polynucleotide hairpin (HP), or when an unbound fluorophore-conjugated polynucleotide (FS) reversibly binds to said first stem region and said loop region of said polynucleotide hairpin (HP); and
a polypeptide with DNA polymerase activity.

In one example, each said first overhang region, said first stem region, said loop region, said second stem region, or said primer binding region comprises one or more nucleotide domains.

In one example, wherein the concentration of the polynucleotide hairpin (HP) is in excess of the concentration of the fluorophore-conjugated polynucleotide (FS).

In one example, wherein said first stem region or second stem region comprises or consists of 14 nucleotides.

In one example, wherein said first stem region or second stem region comprises or consists of about 57% content of G and C nucleotide.

In one example, wherein the polynucleotide primer comprises or consist of 8 to 11 nucleotides.

In one example, wherein said fluorophore moiety is fluorescein amidite (FAM™).

In one example, wherein said quencher is IOWA BLACK™ FQ.

In one aspect there is provided a kit, comprising: a polynucleotide beacon, comprising:
a polynucleotide beacon, comprising:
   a polynucleotide hairpin (HP), comprising: a first overhang region comprising a fluorophore quencher conjugated to the 5′end of said first overhang region, a first stem region positioned 3′ to the first overhang region, a loop region positioned 3′ to the first stem region, a second stem region positioned 3′ to the loop region, such second stem region complementary to the first stem region, and a second overhang region positioned 3′ to the second stem region;
   a fluorophore-conjugated polynucleotide (FS), comprising a fluorophore moiety conjugated to the 3′end of said polynucleotide, said fluorophore-conjugated polynucleotide is adapted to reversibly bind to said first overhang region of said polynucleotide hairpin (HP), and said first stem region and loop region of said polynucleotide hairpin (HP),
a polynucleotide primer configured to reversibly bind to a primer binding region that overlaps with said second stem region and said second overhang region of said polynucleotide hairpin (HP) when a target polynucleotide in a sample reversibly binds to said loop region of said polynucleotide hairpin (HP), or when an unbound fluorophore-conjugated polynucleotide (FS) reversibly binds to said first stem region and said loop region of said polynucleotide hairpin (HP); and
a polypeptide with DNA polymerase activity.

In one example, the polynucleotide primer comprises or consist of 8 to 11 nucleotides.

In one example, wherein said fluorophore moiety is fluorescein amidite (FAM™).

In one example, wherein said quencher is IOWA BLACK™ FQ.

### BRIEF DESCRIPTION OF THE DRAWING

Embodiments of the present disclosure will now be described, by way of example only, with reference to the attached Figures. The application file contains at least one drawing executed in color. Copies of this patent or patent application with color drawings will be provided by the Office upon request and payment of the necessary fee.

FIG. 1 depicts BEAR, which uses an amplifiable beacon, a single enzyme, and a single primer to produce amplified fluorescence signals in response to the presence of a nucleic acid target. The target and an additional fluorescent strand, FS, are displaced. Exponential amplification is enabled by the ability of the displaced target and displaced FS strands to also interact with the amplifiable beacon to initiate the reactions. The small square at the end of the DNA sequences indicates the 5′-end.

FIG. 2 depicts the sequence and domains of the amplifiable beacon. The amplifiable beacon consists of HP in a hairpin conformation with FS bound to its overhang. The 5′ end of HP is conjugated to a quencher and the 3′ end of FS is conjugated to a fluorophore. The close proximity of the fluorophore to the quencher quenches its fluorescence. The complementary base of the MERRF target point mutation in the loop is bolded and underlined.

FIG. 3 depicts the amplifiable beacon formed by annealing HP and FS together. HP folds into a hairpin with a long single-stranded overhang and is conjugated on its 5′ end to a quencher. FS is complementary to the HP overhang and is conjugated to a fluorophore on its 3′ end. The proximity of the quencher to the fluorophore in the amplifiable beacon results in quenched fluorescence. The small square at the end of the DNA sequences indicates the 5′-end.

FIGS. 4A and 4B depict a reaction schematic of BEAR. FIG. 4A shows that binding of the target to the loop and stem of HP (3a, 3b, 2c*) opens the stem to uncage the primer-binding domains (2a*, 2b*). Primer binding and subsequent extension via the polymerase displace the target and the previously annealed FS. The fluorescence of FS is restored as it is separated from the quencher. The target is recycled and thus, amplification is achieved. FIG. 4B shows that the displaced FS also binds to the loop and stem of HP (3a, 2c, 2b) to open the stem and initiate the primer extension. Primer binding and subsequent extension via the polymerase displace both copies of FS. The displaced FS is fluorescent and is also recycled for the amplification reactions. Thus, the cyclic reactions involving both the target and the displaced FS result in the exponential amplification.

FIGS. 5A and 5B depict native PAGE analyses of amplifiable beacon solutions containing various ratios of [HP] to [FS] (1:1.5, 1:1.0, 1:0.85, 1:0.80, 1:0.75, and 1:0.73). The 10% native gel was imaged for fluorescein amidite (FAM™) fluorescence (B), then stained with SYBR™ Gold (A) and imaged again. All products are therefore visualized in FIG. 5A, and only products that have unquenched fluorescence are visualized in FIG. 5B. The bands correspond to: (i) Amplifiable beacon bound with FS, (ii) amplifiable beacon, and (iii) HP. Shown in FIGS. 5A and 5B is the amount of FS relative to HP.

FIG. 6 depicts amplification curves showing the effect of varying the nominal amount of FS (0.85, 0.83, 0.80, 0.78) relative to HP in amplifiable beacon annealing solutions. Target refers to 100 pM MERRF target sequence, and blank refers to all reagents in the BEAR mixture without the MERRF target.

FIG. 7 depicts typical signal amplification curves of BEAR, showing the progression of fluorescence from reactions with either 100 pM of MERRF target or water as blank. The sigmoidal shape of the curve is characteristic of exponential amplification. Fluorescence was normalized against a reporter dye and monitored over time in minutes.

FIGS. 8A and 8B depict BEAR stopped at various time points with chilled EDTA. The reaction components at each time point are separated on a 10% Native PAGE and imaged after (FIG. 8A) SYBR™ gold staining and using the detection of (FIG. 8B) fluorescein amidite (FAM™) fluorescein.

FIG. 9 depicts locations of each mismatch when the clinical negative control (NC) and arbitrary mismatch strands to simulate the effect of mismatches at each location, Mismatch 1 (M1), Mismatch 2 (M2), and Mismatch 3 (M3), are bound to HP. The (x) positioned on each sequence indicates the general location of the mismatch within the strand.

FIGS. 10A and 10B depict the effect on BEAR when using 100 pM of MERRF target (100 pM) or water (Blank) when truncating the primer from 11 nt to 10 nt, 9 nt, or 8 nt from the (FIG. 10A) 5′ end (Primer 5-n) or (FIG. 10B) 3′ end (Primer 3-n). Primer 5-7 yielded no detectable signal in the experimental timeframe.

FIG. 11 depicts Amplification curves showing the effect of temperature (35, 40, 45, 50° C.) on BEAR with 100 pM of MERRF target and blank.

FIG. 12 depicts amplification curves showing the effect of varying Bst 2.0 polymerase concentrations (0.4, 0.2, 0.1, 0.05 U/L) on BEAR with 100 pM of MERRF target and blank.

FIG. 13 depicts amplification curves from reactions of MERRF target at various starting concentrations.

FIG. 14 depicts amplification curves of concentrations from reactions containing low concentrations MERRF target, ranging from 10 pM to 1 fM. Reactions times from 32 min to 56 min were shown. The 8-nt primer was used.

FIG. 15 depicts a comparison between 100 pM of the MERRF target and 100 pM of the negative control against the difference of time at threshold between target and blank (ATt). Error bars represent standard deviation from triplicate analyses.

FIG. 16 depicts the ATt (difference between target and blank time at threshold) of Mismatch locations, the MERRF clinical negative control, and the MERRF target. Reactions initiated with 100 pM of each respective oligonucleotide and performed in triplicates.

FIG. 17 depicts the binding locations of the 11-nt primer (Primer 11) and each truncated primer (Primer 5-n and Primer 3-n) to HP within amplifiable beacons. Stem opening via either the target or FS is not shown. The location of the base in the loop of HP that is complementary to the point mutation in the MERRF target is bolded and underlined.

FIG. 18 depicts representative amplification curves of 10 pM of MERRF target in MCF-7 cell lysate and reagent blank from 32 min to 70 min.

### DETAILED DESCRIPTION

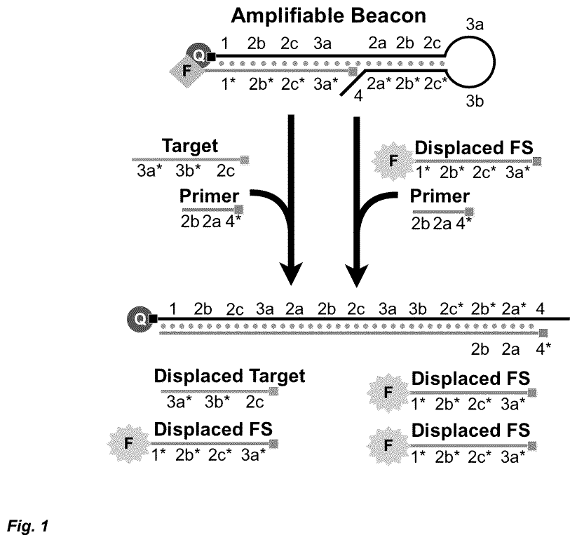

We describe herein a new technique, Beacon-mediated Exponential Amplification Reaction (BEAR), for the detection of nucleic acid targets using isothermal and exponential signal amplification with a single enzyme and a single primer (FIG. 1). Key to the BEAR technique is a specially designed DNA beacon, the amplifiable beacon, that cages a primer-binding region. The target nucleic acid interacts with the amplifiable beacon and opens the stem, uncaging this region for the primer to bind and initiate polymerase extension. The target strand and an additional fluorophore-conjugated strand (FS) are displaced from the beacon and the displaced FS produces a fluorescence signal. The target is recycled and interacts with other amplifiable beacons to initiate additional reactions, which would result in linear amplification. To enable exponential amplification of the detection signal, we designed the displaced FS to also interact with amplifiable beacons to initiate the amplification reaction. Thus, after being displaced, FS serves as a reporter and also initiates more reactions.

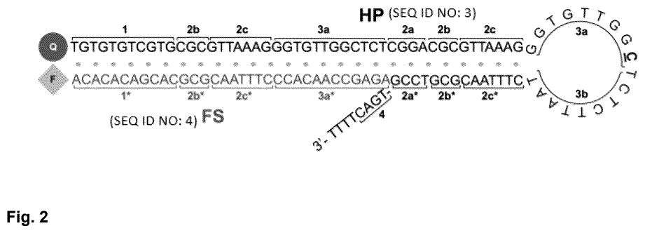

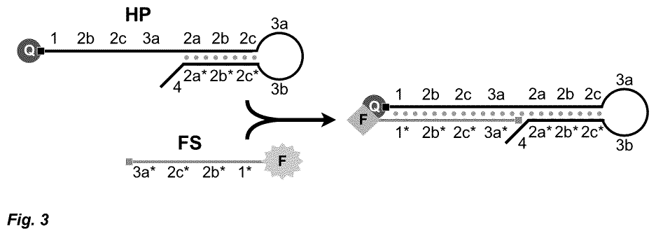

For ease of explanation of our design, we assigned numbers to each nucleic acid domain. Complementary domains are denoted with an asterisk (e.g. domain 1* is complementary to domain 1) (FIG. 2). The amplifiable beacon is created by annealing FS to the long overhang of a DNA hairpin, HP (FIG. 3). A fluorescence quencher is conjugated to the 5′end of HP and a fluorophore is conjugated to the 3′ end of FS. When HP and FS are annealed together to form the beacon, the fluorescence is quenched. The sequence of HP contains functional loop (3a, 3b), stem (2a/2a*, 2b/2b*, 2c/2c*), primer-binding (4, 2a*, 2b*), and long 5′ overhang (1, 2b, 2c, 3a) domains. We designed the 14-bp stem to cage a portion of the primer-binding region, 2a* and 2b*, and thus this region is unavailable for the primer to bind. While 4 is complementary to part of the primer, the main intention of designing this short overhang domain is to prevent extension via the polymerase which would result in target-independent displacement of the FS strand.

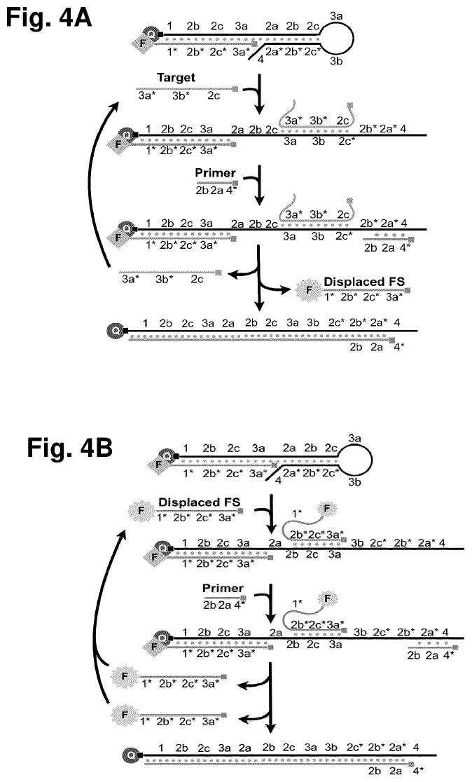

BEAR begins when a nucleic acid target binds to 3a, 3b, and 2c* of the loop and stem of HP within an amplifiable beacon (FIG. 4A). The stem opens and exposes 2a* and 2b* to the primer. The subsequent binding of the primer to 4, 2a*, and 2b* initiates 5′ to 3′ extension via a DNA polymerase, using HP as the template. The polymerase has strong strand displacement activity and displaces the target and the FS that comprised the amplifiable beacon. When FS is displaced from HP, the fluorophore and quencher are separated and the fluorescence is restored. After complete polymerase extension, HP has no further purpose in the reaction and is waste. The displaced target is cycled back to initiate new reactions by binding to other amplifiable beacons (the left-hand portion of FIG. 4).

We achieve exponential amplification of the detection signal by having the displaced FS strands also initiate additional reactions (FIG. 4B). This is because we designed FS to complement both the overhang of HP and the loop and stem of HP. Although the fluorescence of the FS bound to the overhang of HP is quenched, the FS bound to the loop and stem of HP retains its fluorescence. Similar to the reaction initiated by the target, binding of FS to the loop and stem domains (3a, 2c, and 2b) of HP exposes 2a* and 2b* to the primer. The polymerase extends the primer and displaces both the initiating FS and the previously bound FS that comprised the amplifiable beacon, restoring the fluorescence of the latter. Both displaced FS strands can further bind to other beacons to initiate additional reactions. The cyclic reactions initiated by the target and then by the displaced FS results in exponential amplification.

For every reaction, the initiating strand (target or displaced FS from a prior reaction), is recycled and the FS comprising the beacon is displaced, both of which can further initiate more reactions. BEAR plateaus when all amplifiable beacons in solution are exhausted; i.e., where all FS is single-stranded in solution and all HP is converted to waste.

As a proof-of-concept, we applied our technique to detect the point mutation, 8344A>G, in mitochondrial DNA (mtDNA), as a 25-nt target. This point mutation is associated with Myoclonus Epilepsy with Ragged Red Fibres (MERRF). The Seitz group has shown that the conformational constraint of the beacon can discriminate mismatches [1]

For the formation of the amplifiable beacon prior to any target analysis, FS must be bound to the 5′ overhang of HP instead of to the loop and stem, as FS is complementary to both. If FS binds the loop and stem in the absence of the target, the resulting reaction would produce background. To drive the binding of FS to the overhang, we increased the complementarity of FS to the overhang relative to that of the loop and stem by extending the length of 1 on the overhang.

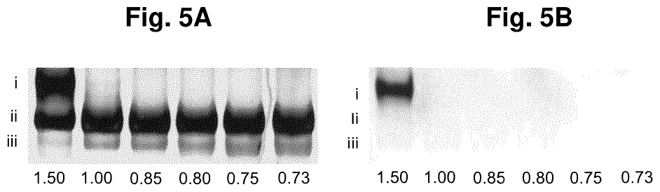

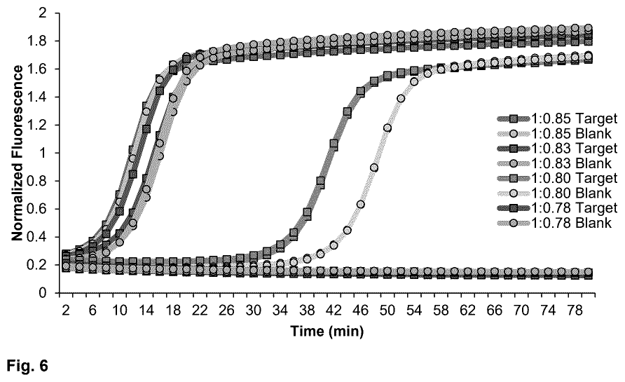

We tested the annealing of FS to HP using various ratios of [HP] to [FS], including 1:1.5, 1:1.0, 1:0.85, 1:0.80, 1:0.75, and 1:0.73. We separated the annealing products from each of these reaction mixtures using polyacrylamide gel electrophoresis (PAGE) (FIG. 5). We first imaged the gel for fluorescein amidite (FAM™) fluorescence (FIG. 5B), then stained the gel using SYBR™ gold (FIG. 5A), which stains both ssDNA and dsDNA. Product (iii) corresponds to HP, and the increasing band intensity corresponds to increasing amounts of HP relative to FS. Product (ii) is the correctly formed amplifiable beacon whose fluorescence is completely quenched. When the amount of FS is in excess of HP in ratio 1.5:1, product (i) is detectable, representing the annealed amplifiable beacon with an additional FS strand bound to the loop and stem. Adding FS in excess of HP in ratio 1.5:1 to force FS to bind the loop and stem of the beacon supports that domain 1 is sufficient to drive the binding of FS to the overhang of HP. We can further optimize the ratios of [HP] to [FS] to maximize amplification of the target while minimizing the background (FIG. 6).

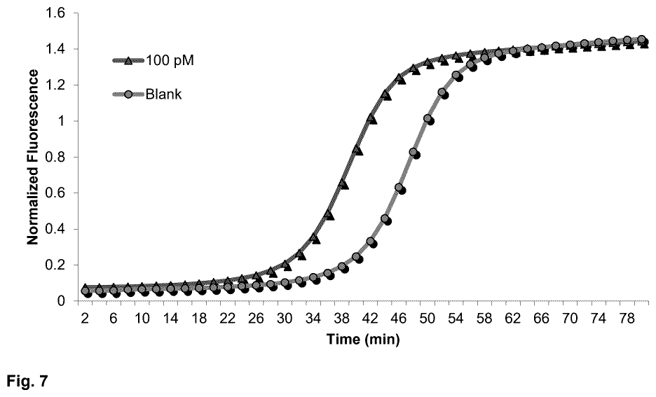

FIG. 7 shows the typical amplification curves of BEAR. The sigmoidal shape of the curves generated by 100 pM of the synthetic MERRF target sequence and blank demonstrate characteristic exponential amplification. Similar to other techniques with exponential amplification, three phases are observed: lag, exponential, and plateau. The reaction at 0 min contains primarily amplifiable beacons, characterizing the lag phase. In the lag phase, there is a small number of initiating molecules (target or displaced FS), which results in the slow propagation of the reaction and fluorescence output below the detection limit of the fluorometer. The exponential phase, at ˜26-54 min, generates substantial fluorescence because of the increased amount of displaced FS. Both the lag phase and exponential phase consist of amplifiable beacons that are readily available in abundance for target or displaced FS to bind to for the propagation of the reaction. The plateau phase is a result of the exhaustion of available amplifiable beacons. In the plateau phase, almost all the amplifiable beacons are converted to displaced FS and waste.

The background signal may arise from the leakage of our DNA structures as a result of incomplete chemical synthesis. Leakage may cause improper formation of amplifiable beacon and may result in target-independent displacement of FS that is then exponentially amplified. We have considered various sources of background (Section 8, Supporting Information herein) and minimized background in the optimization process.

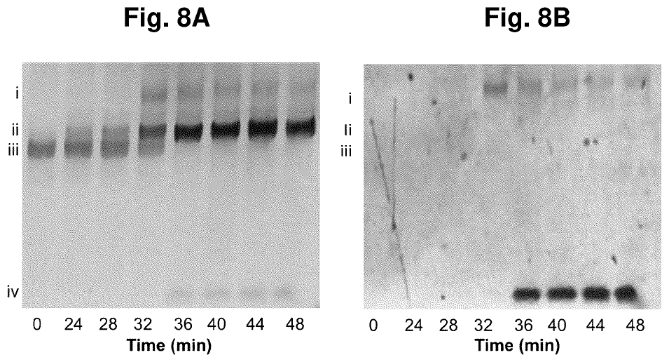

We detected the amplification products at various amplification time points by sampling the reaction mixture and separating the components using PAGE (FIG. 8). The gel was imaged for fluorescein amidite (FAM™) fluorescence (FIG. 8B) and SYBR™ gold staining (FIG. 8A). The amplifiable beacon and reaction products, including the displaced FS, transient complexes, and waste, were detected. The predicted products were detected in each phase: lag (0-28 min), exponential (˜32-46 min), and plateau (after 48 min).

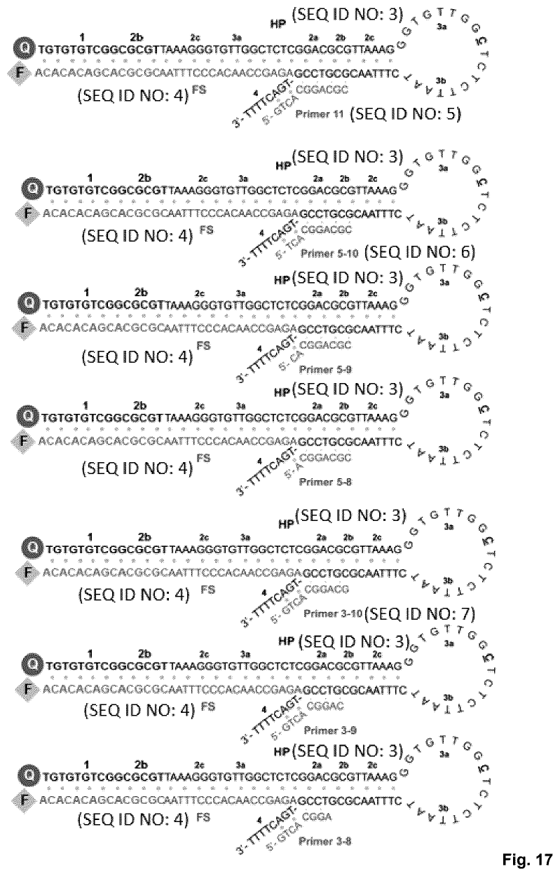

The primer binding location on HP is partially caged within the stem, 2b* and 2a*, and partially exposed, 4. To determine the optimal primer length, we designed two sets of primers (Section 5, Supporting Information herein). In one set, we fixed the sequence of the primer on the 5′ end (Primer 5-n) and changed the length of the domain complementary to the stem of HP (complement to 2b* and 2a*). In the second set, we fixed the sequence of the primer on the 3′ end (Primer 3-n) and changed the length of 4* (FIG. 17). We also tested a full-length primer of 11-nt (Primer 11).

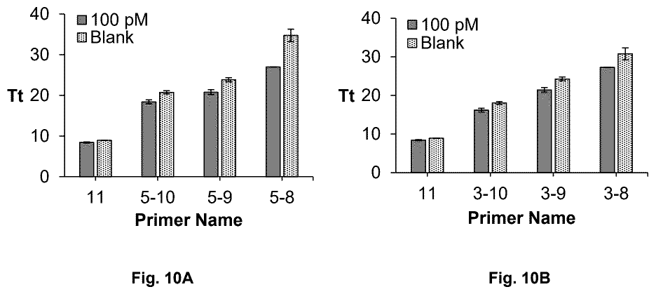

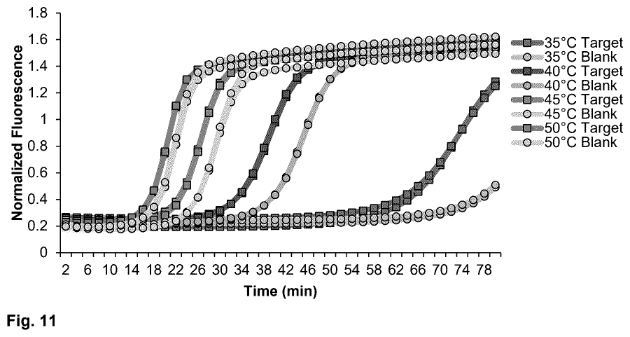

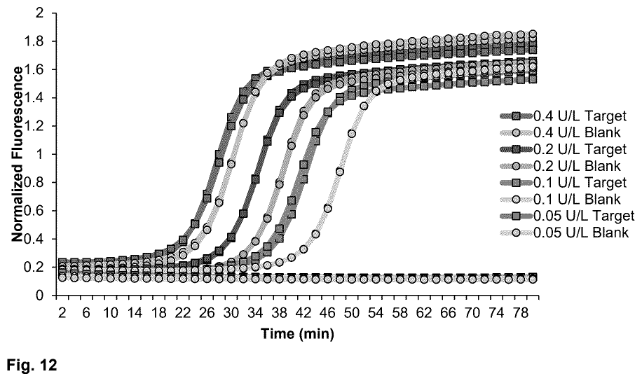

The results obtained from using the various types of primers are shown in FIG. 10A, B. The full-length primer, Primer 11, produced a fast reaction in both the target and the blank resulting in a small ΔTt (difference between Tt_{blank} and Tt_{target}). As expected, both primer sets resulted in slower reactions times with decreasing primer lengths, which implies that the duplex stability of the bound primer contributes to the rate of the reaction. The shortest primer in each set produced the largest ΔTt. Primers 5-8 and 3-8 showed no substantial difference in ΔTt, indicating that the binding position of the primer does not substantially affect ΔTt. For subsequent experiments, we used Primer 5-8. Other reaction parameters optimized included the ratio of HP to FS in forming the amplifiable beacon (FIG. 6), reaction temperature (FIG. 11), polymerase concentrations (FIG. 12), complementary domain (Section 9, Supporting Information herein), and buffer concentrations.

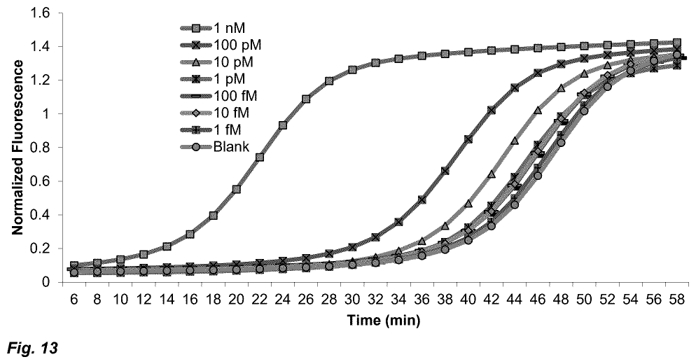

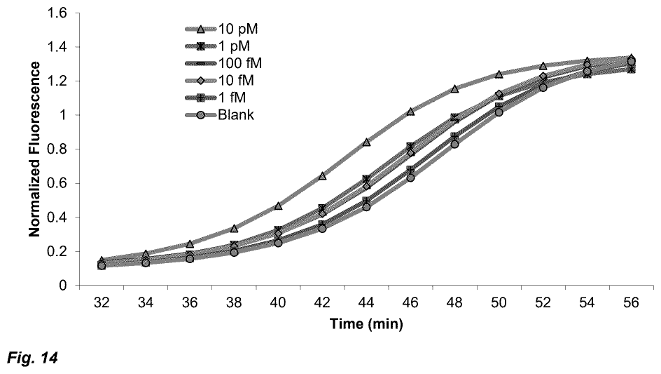

We next assessed the dynamic range of our BEAR using various concentrations of the MERRF target sequence, from 1 nM to 1 fM (FIG. 13 and FIG. 14). We also obtained the limit of detection (LOD) by testing seven replicate blanks and comparing it to our calibration. The LOD, defined as the concentration equivalent to three times the standard deviation of blanks plus the background signal (3SD_{blank}+Tt_{blank}), was about 10 fM.

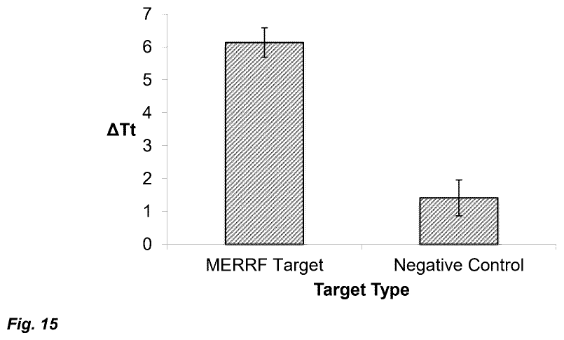

We also investigated the specificity of BEAR by assessing its detection of the MERRF target sequence and the clinical negative control (NC), the normal mtDNA sequence. We compared the reaction of 100 pM of the MERRF target with 100 pM of the negative control (FIG. 15). BEAR achieved a discrimination factor (DF) of 16. Further analysis of target and mismatch binding region placement is described herein.

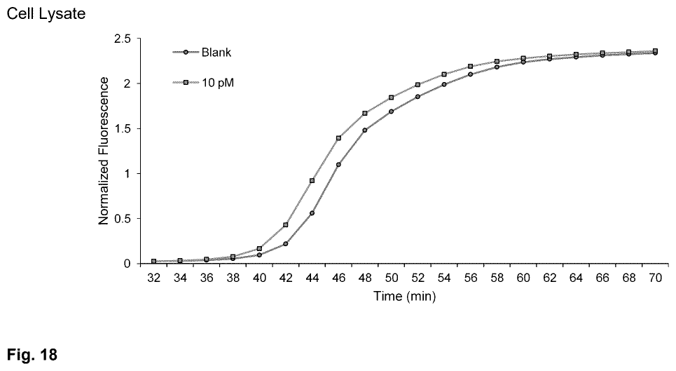

We applied the BEAR technique to the detection of the 10 pM MERRF target in cell lysate (FIG. 18—And Section 10 of Supporting Information). The measured concentration was 9.1±0.8 pM, consistent with the expected concentration and representing an average recovery of 91%.

Our results demonstrate that with a single enzyme and a single primer the BEAR technique is able to achieve isothermal and exponential amplification of the detection signal for a clinically relevant mutation target. Using a single polymerase and a single primer simplifies the technical procedure, which is valuable for point-of-care testing and on-site analysis. BEAR is particularly appropriate for the detection of short nucleic acid targets, as we have demonstrated the detection of a 25-nt MERRF target. BEAR may also be useful for the detection of microRNA (miRNA). The short target length of miRNAs poses challenges in accommodating more than one primer as is required by other techniques. In some examples, BEAR may be used to detect other types of target polynucleotides, including but not limited to RNA, mRNA, aptamers, mtDNA, and siRNA. In preferred embodiments, the target polynucleotide is any short length nucleic acid, including those that are less than 50, 40, or 30 nucleotides in length.

In one aspect there is provided a Beacon-mediated Exponential Amplification

Reaction (BEAR) system, comprising:
a polynucleotide beacon, comprising:
   a polynucleotide hairpin (HP), comprising: a first overhang region comprising a fluorophore quencher conjugated to the 5′end of said first overhang region, a first stem region positioned 3′ to the first overhang region, a loop region positioned 3′ to the first stem region, a second stem region positioned 3′ to the loop region, such second stem region complementary to the first stem region, and a second overhang region positioned 3′ to the second stem region;
   a fluorophore-conjugated polynucleotide (FS), comprising a fluorophore moiety conjugated to the 3′end of said polynucleotide, said fluorophore-conjugated polynucleotide is adapted to reversibly bind to said first overhang region of said polynucleotide hairpin (HP), and said first stem region and loop region of said polynucleotide hairpin (HP),
a polynucleotide primer configured to reversibly bind to a primer binding region that overlaps with said second stem region and said second overhang region of said polynucleotide hairpin (HP) when a target polynucleotide in a sample reversibly binds to said loop region of said polynucleotide hairpin (HP), or when an unbound fluorophore-conjugated polynucleotide (FS) reversibly binds to said first stem region and said loop region of said polynucleotide hairpin (HP); and
a polypeptide with DNA polymerase activity.

In one example, the polypeptide with DNA polymerase activity is Bst 2.0. In preferred embodiments, the polypeptide with DNA polymerase activity is a polymerase with strand displacement activity. Non-limiting examples include Bst 3.0 and Large Klenow Fragment.

The term “fluorophore,” as used herein refers to a composition that is inherently fluorescent or demonstrates a change in fluorescence upon binding to a biological compound or metal ion, i.e., fluorogenic. Fluorophores may contain substituents that alter the solubility, spectral properties or physical properties of the fluorophore. Numerous fluorophores are known to those skilled in the art and include, but are not limited to coumarin, cyanine, benzofuran, a quinoline, a quinazolinone, an indole, a benzazole, a borapolyazaindacene and xanthenes including fluorescein, rhodamine and rhodol as well as semiconductor nanocrystals and other fluorophores. In one example, the fluorophore is fluorescein amidite (FAM™).

In one example, the quencher is the Dark Quencher IOWA BLACK™ FQ. The quencher may be any species that quenches the fluorescence of the fluorophore on the fluorophore-conjugated polynucleotide (FS) when bound to the polynucleotide hairpin (HP).

The term “polypeptide” may also be referred to as “protein” and as used herein corresponds to an amino acid polymer. This includes the proteins, protein fragments, genetically modified proteins, oligopeptides and analogs thereof.

Samples, which may also refer to as biological samples, from a subject include, but are not limited to bodily fluids.

As used herein the term “bodily fluid” refers to any fluid found in the body of which a sample can be taken for analysis. Non-limiting examples of bodily fluids include blood, plasma, serum, lymph, sudor, saliva, tears, sperm, vaginal fluid, faeces, urine or cerebrospinal fluid.

Biological samples from a subject also includes samples derived, e.g., by biopsy, from cells, tissues or organs. This also encompasses samples comprising subcellular compartments or organelles, such as the mitochondria, Golgi network or peroxisomes. Biological samples also encompass gaseous samples, such as volatiles of an organism. Biological samples may be derived from a subject.

Techniques for obtaining different types of biological samples are well known.

Sample can be obtained by conventional methods, using processes known in the state of the art by the person skilled in the art. In some examples, a sample is used directly (e.g., fresh or frozen), or can be manipulated prior to use, for example, by extraction (for example of nucleic acids), fixation (e.g., using formalin) and/or embedding in wax (such as FFPE tissue samples).

Biological samples may be pre-treated before use. Pre-treatment may include treatments required to release or separate the compounds or to remove excessive material or waste. Suitable techniques comprise centrifugation, extraction, fractioning, purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art.

The sample may be from a subject.

The term “subject” as used herein, refers to any mammal or non-mammal that would benefit from determining the benefit from treatment, treatment, diagnosis, therapeutic monitoring and/or prognosis. In certain examples a subject or patient includes, but is not limited to, humans, farm animals, companion animals (such as cats, dogs and horses), primates and rodent (such as mice and rats). In a specific embodiment, the subject is a human. The subject may be an infant, an adolescent, or an adult.

In one example, the sample comprises a target polynucleotide.

The terms “nucleic acid,” “polynucleotides,” and “oligonucleotides” refers to biopolymers of nucleotides and, unless the context indicates otherwise, includes modified and unmodified nucleotides, and both DNA and RNA.

The term “primer”, “oligonucleotide”, “oligonucleotide primer”, or “polynucleotide primer” refers to a short polynucleotide that satisfies the requirements that it must be able to form complementary base pairing sufficient to anneal to a desired nucleic acid template, in order to initiate nucleic acid synthesis using a suitable DNA polymerase. It will be appreciated that the primer backbone is not necessarily limited to the one via phosphodiester linkages. For example, it may be composed of a phosphothioate derivative having S in place of O as a backbone or a peptide nucleic acid based on peptide linkages. The bases may be those capable of complementary base pairing.

The oligonucleotide primers, fluorophore-conjugated polynucleotide (FS), and polynucleotide hairpin (HP) are generally isolated.

The terms isolated as used herein generally refers to a biological component (such as a nucleic acid) that has been substantially separated or purified away from biological or other components. Nucleic acids that have been “isolated” include nucleic acids purified by standard purification methods. The term also embraces nucleic acids prepared by recombinant expression in a host cell and subsequently purified, as well as chemically synthesized nucleic acid molecules. Isolated does not require absolute purity, and can include nucleic acid molecules that are at least 50% isolated, such as at least 75%, 80%, 90%, 95%, 98%, 99%, or even 99.9% isolated. An isolated nucleic acid may be in solution (e.g., water or an aqueous solution) or dried.

Further experimental details can be found in Supporting Information.

### Supporting Information

### Table of Contents

1) Experimental Procedures
2) Specific Design Parameters
3) Amplifiable Beacon: Ratios of HP: FS
4) Reaction Progression
5) Primers
6) Effect of varying conditions
7) Dynamic Curve: Low Concentrations
8) Sources of Background
9) Target Mismatch Placement
10) Detection of MERRF Target in Cell Lysate
11) References

### 1. EXPERIMENTAL PROCEDURES

### Preparation of DNA Oligonucleotides

Sequences of the hairpin probe (HP) and fluorophore-conjugated stand (FS) are listed in Table 1. All DNA oligonucleotides (oligos) were synthesized and purified by Integrated DNA Technologies (IDT, Coralville, IA). Oligos HP and FS were resuspended to 30 M using 20 mM TrisHCl pH 7.4. Target oligos (MERRF target, negative control, and mismatches) and primers were resuspended to 100 μM using 20 mM TrisHCl (pH 7.4), and Milli-Q ultrapure water. The stock oligonucleotides were stored at −18° C.

### Preparation of the Amplifiable Beacon

Solutions of HP and FS mixtures to anneal the amplifiable beacon were prepared at room temperature. The solutions contained 5 M HP, varying concentrations of FS depending on the desired HP: FS ratio, 250 mM MgCl₂, 20 mM TrisHCl (pH 7.4), and Milli-Q ultrapure water. Solutions were slowly cooled in a benchtop thermocycler (Biorad, USA) from 90° C. to 20° C. in 2 hr. Annealed amplifiable beacon solutions were stored at 4° C. for up to two weeks.

### Procedures of Beacon-Mediated Exponential Amplification Reaction (BEAR) and Fluorescence Detection

Reaction solutions were prepared at room temperature in a master mix and typically contained 100 nM of annealed amplifiable beacon solution, 0.1 U/μL Bst 2.0 DNA Polymerase (New England Biolabs, Whitby, ON, Canada), 500 nM primer, 200 UM Deoxynucleotide Solution Mix (dNTP) (New England Biolabs, Whitby, ON, Canada), 1×ROX Reference Dye (Thermofisher Scientific, Canada), 1× Isothermal Buffer that contained 20 mM Tris-HCl, 10 mM (NH₄)₂SO₄, and 50 mM KCl, 2 mM MgSO₄, 0.1% TWEEN® 20 (pH 8.8) (New England Biolabs, Whitby, ON, Canada), and Milli-Q ultrapure water. Reaction solutions were added to 2 μL of either MERRF target sequence, negative control, mismatch target or Milli-Q ultrapure water for a total volume of 20 μL per reaction and incubated at 40° C. in STEPONEPLUS® Real-Time PCR System (Thermofisher Scientific, Canada) for isothermal fluorescence detection every 2 min for a total reaction run time of 80 min. Fluorescence intensity was normalized against a reference dye (Normalized Fluorescence), ROX. Normalized fluorescence was calculated by dividing the fluorescence emission of the reporter probe, FS, by that of the reference dye, ROX Reference Dye. Thresholds were set by the determination of a baseline by measuring the initial fluorescence in all samples prior to detection of an increase in fluorescence and multiplying the average value by 10 standard deviations.

Gel electrophoresis was performed using 10% Native PAGE and run at 80V for 1.5 hours at room temperature using 2× the concentrations of reagents.

### Mcf-7 Cell Culture and Preparation of Cell Lysate

MCF-7 adherent epithelial cells were cultured in 50 mL T-25 vented flasks treated for cell cultures. The DMEM medium was supplemented with 10% FBS and 1% of penicillin/streptomycin. Cells were grown in 5% CO₂ at 37° C. with 90% humidity. Cells were sub-cultured passaging every 3-4 days at 85-90% confluency using 0.25% (w/v) trypsin-EDTA to detach the cells. Detached cells were centrifuged, resuspended and added into new flasks with fresh, pre-warmed media.

Cells were detached from flasks, counted, and diluted according to the protocols by Osborn et al. [2] where 80 μL of Tris-HCl (pH 7.4) was added for every 10⁷ cells. The cell suspension was sonicated to lyse cells and checked for complete lysis visually under microscope. The suspension was centrifuged for 14 min at 13000 rpm (16200×g rcf) to remove cellular debris, and the supernatant was saved. The supernatant was heated to 65° C. for 10 min to inactivate DNAses. The resulting solution was stored in 4° C. and used as cell lysate in subsequent experiments (Section 10 of Supplementary Information).

TABLE 1
Sequences of DNA oligonucleotides

| Name of | | SEQ |
|---|---|---|
| Oligo- | Sequence of | ID |
| nucleotide | Oligonucleotide (5′-3′) | NO: |
| MERRF | GTT AAA GAT TAA GAG AGC CAA | 1 |
| Target | CAC CAA A | |
| Negative | GTT AAA GAT TAA GAG AAC CAA | 2 |
| Control | CAC CAA A | |
| | Iowa Black ™ FQ-TGT GTG TCG TGC | 3 |
| | GCG TTA AAG GGT GT TGG CTC | |
| HP | TCG GAC GCG TTA AAG GGT GTT | |
| | GGC TCT CTT AAT CTT TAA CGC | |
| | GTC CGT GAC TTT T | |
| FS | AGA GCC AAC ACC CTT TAA CGC | 4 |
| | GCA CGA CAC ACA-FAM ™ | |

### 2. SPECIFIC DESIGN PARAMETERS

Careful design of our oligonucleotides is important for the correct formation of the amplifiable beacon and the efficiency of the technique. This section will outline important considerations for designing FS and HP. FIG. 2 shows the sequences of HP and FS forming the amplifiable beacon separated into domains.

FS is responsible for enabling exponential amplification of the detection signal. In BEAR, one amplifiable beacon releases two reaction initiators, the displaced target and the displaced FS. Therefore, FS must be designed to bind to both the overhang of HP to form the amplifiable beacon and the loop and stem domains to initiate BEAR. As a result, FS contains complementary regions to both the overhang of HP and the loop and stem region of HP.

To drive FS to preferentially bind to the overhang instead of the loop and stem for the correct formation of the amplifiable beacon, we added domain 1 on the overhang (and 1* on FS) to increase the melting temperature of the duplex forming the stable amplifiable beacon. As domain 1 is not dependent on target sequence, the sequence and length can be adjusted to tune the desired melting temperature of the duplex formed between FS and the overhang of HP. We designed the complementary domains of 1/1* to increase the melting temperature of the FS: HP duplex by about 25° C. versus the loop toehold, 3a/3a*.

The stem of HP must also be designed to balance the speed of the reaction while ensuring amplifiable beacons do not open in the absence of the target. Increasing the length of the stem and increasing GC content will increase the likelihood that the stem does not open in the absence of any reaction initiators. However, too long of a stem and too high of GC content may inhibit or slow the reaction. We designed the stem to be 14-bp with 57% GC content.

HP has a 3′ overhang of four T residues. The function of this overhang is to prevent polymerase extension of the stem using the 5′ overhang as a template. The 3′ overhang also provides additional nucleotides upstream of the primer for polymerase binding.

Another important design consideration is the location of the 25-nt MERRF target when bound to the loop and stem of HP. We aimed to minimize the ability of the negative control sequence, the clinically healthy sequence, to open the stem. The complementary sequence to capture the MERRF target was oriented such that when bound to the amplifiable beacon, the location of the point mutation was placed in the centre of the loop with 8-nt and 9-nt flanking either side of the point mutation location. Further discussion is provided in Section 9 of the Supporting Information, Target Mismatch Placement. Additionally, we designed the target to have a 3-nt long 3′ unhybridized region when bound to its complementary domains on the loop and stem of the amplifiable beacon. The function of this unhybridized region on the target is to inhibit the polymerase from extending the target as if it were a primer.

FIG. 2 depicts the sequence and domains of the amplifiable beacon. The amplifiable beacon consists of HP in a hairpin conformation with FS bound to its overhang. The 5′ end of HP is conjugated to a quencher and the 3′ end of FS is conjugated to a fluorophore. The close proximity of the fluorophore to the quencher quenches its fluorescence. The complementary base of the MERRF target point mutation in the loop is bolded and underlined.

### 3. AMPLIFIABLE BEACON: RATIOS OF HP TO FS

The amplifiable beacon was prepared by mixing 5 UM of HP and various amounts of FS in a single tube with annealing solution and heat denatured at 95° C. for 5 min, followed by slow cooling to room temperature in 2 hr. Denaturation of HP and FS converts any dsDNA secondary structures into ssDNA. Slow cooling the solution to room temperature results in HP folding into a hairpin first, followed by FS annealing to the 5′ overhang of HP. FS preferentially binds to the overhang of HP instead of the loop and stem of HP due to the greater duplex stability attributed to the additional 1/1* domain interaction. Although it is possible that HP can dimerize because of the nature of the hairpin stem, the domains that make up the stem are too short (14-bp) to withstand the initial denaturation and the formation of the more favourable intrastrand hairpin conformation will precede over HP interstrand dimerization.

The ratio of HP to FS is important when annealing the amplifiable beacon. In ratios with an excess of HP to FS, the resulting solution contains amplifiable beacon and HP in a hairpin conformation (FIGS. 5A&B). In ratios with an excess of FS to HP, the resulting solution contains amplifiable beacons bound to FS. High amounts of FS in annealing mixes may lead to the formation of amplifiable beacons with an additional FS bound to the loop and/or stem. This is undesirable because the additional FS bound to amplifiable beacons can initiate BEAR in the absence of the target, thereby increasing the background signal. Our strategy to prevent background due to FS binding to amplifiable beacons in the annealing process is to use annealing solutions where HP is always in excess of FS. Adjusting the ratios of [HP] to [FS] can control the correct formation of the amplifiable beacon and can be further analyzed by reaction with the target and blank in BEAR.

FIG. 6 shows the effect on BEAR when using amplifiable beacon annealed from various ratios of [HP] to [FS]. Nominal ratios 1:0.85 and 1:0.83 are presumed to have de facto excess FS relative to HP, as both of these ratios show an increase in both the reaction and blank signal, where the target-initiated reaction and blank signal become indistinguishable. On the other hand, if HP is in excess, there would be some free HP with its overhang not hybridized to FS. Because the complementarity of FS to the 5′ overhang is more favourable than that of the loop and stem of HP, it is likely that the displaced FS from the target-initiated reaction will preferentially bind to the free 5′ overhang on HP instead of the loop, which halts the propagation of the reaction. We suspect that the lag time prior to the exponential amplification for each of the curves may partially be due to displaced FS binding to the overhang of free HP in solution, rather than the loops and stems of HP within amplifiable beacons, resulting in linear amplification. This prediction is supported b the results obtained with excess of HP over FS. With an HP: FS ratio of 1:0.75, the high HP concentration relative to FS does not produce detectable exponential amplification for the duration of the 80 min. The annealing solution corresponding to a nominal ratio of 1:0.80 of HP to FS maximized the amount of the correctly formed amplifiable beacon without increasing the blank signal substantially and was used for subsequent reactions.

FIG. 6 depicts amplification curves showing the effect of varying the nominal amount of FS (0.85, 0.83, 0.80, 0.78) relative to HP in amplifiable beacon annealing solutions. Target refers to 100 pM MERRF target sequence, and blank refers to all reagents in the BEAR mixture without the MERRF target.

### 4. REACTION PROGRESSION

The typical amplification curve of BEAR, shown in FIG. 7, can be split into three phases. The phases include lag (0-26 min), exponential (26 min-54 min) and plateau (>54 min). The lag phase consists of an abundance of available amplifiable beacons. The signal output generated in the lag phase is below the detection limits. The exponential phase consists of many displaced target and FS that are continually reacting with other amplifiable beacons to produce detectable fluorescence signals and more displaced FS. The plateau phase is a result of the exhaustion of amplifiable beacons. In the plateau, all amplifiable beacons are converted to waste. Because there are no available amplifiable beacons for FS to react with, FS is free in solution.

FIG. 7 depicts typical signal amplification curves of BEAR, showing the progression of fluorescence from reactions with either 100 pM of MERRF target or water as blank. The sigmoidal shape of the curve is characteristic of exponential amplification. Fluorescence was normalized against a reporter dye and monitored over time in minutes.

FIG. 8 shows BEAR amplification products at various time points separated by PAGE. At 0 min, the only apparent band corresponds to the annealed amplifiable beacon structure (iii). This band disappears as the reaction proceeds and yields no fluorescence. Waste (ii), a higher molecular weight than the amplifiable beacon, increases throughout the reaction and does not fluoresce. Both displaced FS and FS bound to the loop/stem of HP within amplifiable beacons retain fluorescence. Thus, the fluorescing product at 46-48 min corresponds to the high molecular weight complex of amplifiable beacons bound to FS. Amplifiable beacons bound to FS appears in the exponential phase but disappears as the reaction proceeds. The appearance and disappearance of this transient structure is consistent with our scheme. Low molecular weight, free FS (iv), can be seen fluorescing from 36-48 min when there the amount of waste is greater than that of amplifiable beacons.

In FIG. 8, BEAR was stopped at various time points with chilled EDTA. The reaction components at each time point are separated on a 10% Native PAGE and imaged after (A) SYBR™ gold staining and using the detection of (B) fluorescein amidite (FAM™) fluorescein.

### 5. PRIMERS

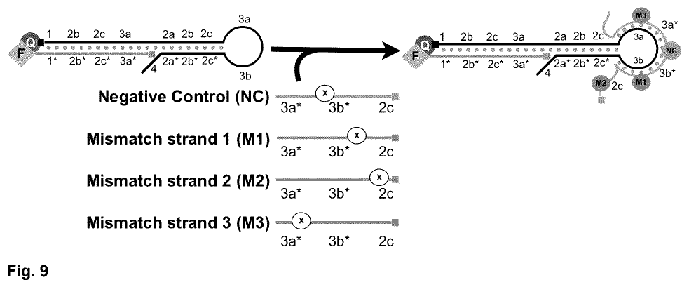

Various primer lengths and primer binding positions were tested. Two sets of primers were designed. In one set, the sequence of the primer on the 5′ end was fixed and changed the length of the domain complementary to the stem of HP. This set is referred to as “Primer 5-n”, where n is the number of nucleotides making up the primer. In the second set of primers, the sequence of the primer on the 3′ end was fixed and the length of the domain complementary to 4 on HP was changed. This set is referred to as Primer “3-n”. The names and sequences of each primer used are shown in Table 2, and the binding positions of each primer are shown in FIG. 9. Primer 11 refers to the full length primer. The effect of each primer on BEAR is shown in FIG. 10A and FIG. 10B and are discussed in the main text.

TABLE 2
Primer sequences with the original, 9 nt
primer,and variations in length and position.

| Name of | Sequence of | SEQ |
|---|---|---|
| Oligonucleo- | Oligonucleotide | ID |
| tide | (5′-3′) | NO: |
| Primer 11 | GTCACGGACGC | 5 |
| Primer 5-10 | TCACGGACGC | 6 |
| Primer 5-9 | CACGGACGC | |
| Primer 5-8 | ACGGACGC | |
| Primer 5-7 | CGGACGC | |
| Primer 3-10 | GTCACGGACG | 7 |
| Primer 3-9 | GTCACGGAC | |
| Primer 3-8 | GTCACGGA | |

FIG. 17 depicts binding locations of the 11-nt primer (Primer 11) and each truncated primer (Primer 5-n and Primer 3-n) to HP within amplifiable beacons. Stem opening via either the target or FS is not shown. The location of the base in the loop of HP that is complementary to the point mutation in the MERRF target is bolded and underlined.

FIG. 10A, B depicts the effect on BEAR when using 100 pM of MERRF target (100 pM) or water (Blank) when truncating the primer from 11 nt to 10 nt, 9 nt, or 8 nt from the (A) 5′ end (Primer 5-n) or (B) 3′ end (Primer 3-n). Primer 5-7 yielded no detectable signal in the experimental timeframe.

### 6. EFFECT OF VARYING CONDITIONS

The rate of the BEAR can be increased or decreased by varying the temperature and concentration of polymerase used. FIG. 11 and FIG. 12 show that although the reaction is increased at higher temperatures or increased concentrations of polymerase, respectively, the background is also increased. Reaction conditions of a temperature of 40° C. with 0.1 U/L of Bst 2.0 polymerase for resulted in the most optimal target to blank signal ratio in <1 hr.

FIG. 11 depicts amplification curves showing the effect of temperature (35, 40, 45, 50° C.) on BEAR with 100 pM of MERRF target and blank.

FIG. 12 depicts amplification curves showing the effect of varying Bst 2.0 polymerase concentrations (0.4, 0.2, 0.1, 0.05 U/L) on BEAR with 100 pM of MERRF target and blank.

FIG. 13 depicts amplification curves from reactions of MERRF target at various starting concentrations.

### 7. DYNAMIC CURVE: LOW CONCENTRATIONS

The dynamic range of BEAR was tested using various concentrations of the MERRF target (FIG. 13). The curves of the low concentrations (10 pM to 1 fM) are shown in FIG. 14 from 32 to 56 min.

FIG. 14 depicts amplification curves of concentrations from reactions containing low concentrations MERRF target, ranging from 10 pM to 1 fM. Reactions times from 32 min to 56 min were shown. The 8-nt primer was used.

### 8. SOURCES OF BACKGROUND

Similar to other strategies with exponential amplification, any small background can be exponentially amplified as well. We considered the various sources of background and attempted to minimize them in our optimization processes. The background may come from two main sources: (1) malformation of the HP stem and (2) FS bound to the loop of amplifiable beacons. Malformation of the HP stem may arise in events such as base mismatch or DNA synthesis errors. We have attempted to reduce the contribution of background from stem malformation through careful design of the stem sequence and through denaturation followed by the 2 hr slow cooling process to create the amplifiable beacon. We also chose the primer length and position combination that produced the best signal to background ratio.

Secondly, FS bound to the loop of amplifiable beacons can increase background. In the design of HP (Supporting Information), inclusion of Domain 1 helps to drive the binding of FS to the overhang of HP to form the amplifiable beacon. When forming the amplifiable beacon, FS is mixed with an excess amount of HP. However, it is possible that even if HP is in excess, some FS may bind the loop and stem of HP in amplifiable beacons, which initiates the reaction and increases the background. We have minimized the contribution to background from this source by optimizing the ratio of HP to FS when annealing the amplifiable beacon prior to target analysis.

### 9. TARGET MISMATCH PLACEMENT

Our BEAR technique for the detection of the 25-nt nucleic acid sequence corresponding to the 8344A>G point mutation resulting in MERRF situates the location of the point mutation in the centre of the loop of HP when the target is bound and therefore, the clinical negative control (Negative Control) contains a mismatch at this location. The location of the mismatch can affect the specificity of the reaction by its ability to open the stem after binding to the loop. To test the optimal location of the point mutation, we tested our MERRF target, Negative Control and three mismatches located on either side of the loop, and on the stem nearest to the 3′ end of HP. To do this, we designed three arbitrary mismatches (Mismatch 1, 2, and 3) in the MERRF target sequence. The sequences for the MERRF target, negative control, and arbitrary mismatches are listed in Table 3 and the locations when bound to the amplifiable beacon are shown in FIG. 9.

Table 3. Sequences of the MERRF target A8344G mutation in comparison to the clinical negative control (wildtype) and other mismatches tested to determine the optimal complementary region on HP for the position of the point mutation. Location of MERRF point mutation is underlined. Arbitrary mismatches created are bolded.

TABLE 3

| | Mismatch | | |
|---|---|---|---|
| Name of | Position | Sequence of | SEQ |
| Oligonucle- | Relative | Oligonucleo- | ID |
| otide | to HP | tide (5′-3′) | NO: |
| MERRF | Centre of | GTTAAAGATTAAGA | 8 |
| Target | loop | GAGCCAACACCAAA | |
| Negative | Centre of | GTTAAAGATTAAGA | 9 |
| Control | loop | GAACCAACACCAAA | |
| Mismatch 1 | On loop | GTTAAAGATGAAGA | 10 |
| | near HP 3′ | GAGCCAACACCAAA | |
| | end | | |
| Mismatch 2 | On stem | GTTGAAGATTAAGA | 11 |
| | near HP 3′ | GAGCCAACACCAAA | |
| | end | | |
| Mismatch 3 | On loop | GTTAAAGATTAAGA | 12 |
| | near HP 5′ | GAGCCAAGACCAAA | |
| | end | | |

FIG. 9. Locations of each mismatch when the clinical negative control (NC) and arbitrary mismatch strands to simulate the effect of mismatches at each location, Mismatch 1 (M1), Mismatch 2 (M2), and Mismatch 3 (M3), are bound to HP. The (x) positioned on each sequence indicates the general location of the mismatch within the strand.

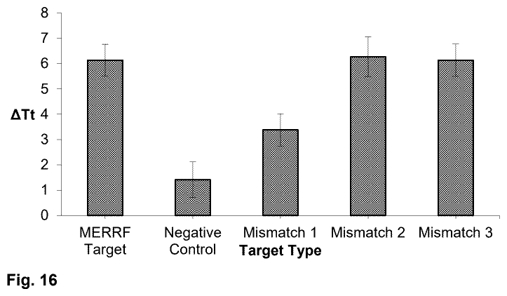

The ΔTt resulting from 100 pM of MERRF target, Negative Control, and the three mismatches (Mismatch 1-3) are shown in FIG. 16. A large ΔTt for the MERRF target, and low ΔTt for the Negative Control is desirable for specificity. Aside from the Negative Control, Mismatch 1 produced the largest ΔTt. This mismatch was placed on the loop adjacent to the stem binding domain, 2c. Mismatch 2, which places the mismatch within 2c, yielded a similar ΔTt to the MERRF target. Because this mismatch has the complete loop toehold complementary sequence, it is able to bind to the loop as does the MERRF target, yielding poor specificity. Mismatch 3 also produced ΔTt similar to that of the MERRF target. When comparing Mismatch 1 and 3, Mismatch 1 was more effective in producing better specificity than that of Mismatch 3, indicating that the position of the mismatch on the loop relative to the stem is an important factor in opening the stem. Mismatch 1 is located adjacent to the stem complementary domain 2c, which may have inhibited stem opening. Thus, as Mismatch 3 was placed further away from 2c, it allowed complete binding to the loop adjacent to the stem domain and as a result, stem opening for the initiation of the reaction. The Negative Control yielded the most optimal specificity as the point mutation was placed in the centre of the loop. This location reduced the ability for the Negative Control to effectively bind the loop toehold to initiate the reaction.

FIG. 16. The ΔTt (difference between target and blank time at threshold) of Mismatch locations, the MERRF clinical negative control, and the MERRF target. Reactions initiated with 100 pM of each respective oligonucleotide and performed in triplicates.

### 10. DETECTION OF THE MERRF TARGET IN CELL LYSATE

Because mitochondrial DNA mutations can occur in epithelial cells, we tested detection of the MERRF target DNA spiked in the lysate of MCF-7 human epithelial cells. A measured amount of MERRF target DNA was added to the lysate of 10⁷ MCF-7 human epithelial cells so that the concentration of the MERRF target DNA was 10 pM. Representative amplification curves from the detection of 10 pM MERRF target DNA in the lysate of MCF-7 human epithelial cells and from the reagent blank are shown in FIG. 18. Triplicate analyses of the cell lysate, using a calibration of the MERRF target DNA in buffer solutions, determined that the concentration of the MERRF DNA was 9.1±0.8 pM. These results, representing an average recovery of 91%, are consistent with the expected concentration of 10 pM. No MERRF DNA was detectable in the cell lysate without the addition of the MERRF DNA.

FIG. 18 depicts representative amplification curves of 10 pM of MERRF target in MCF-7 cell lysate and reagent blank from 32 min to 70 min.

### 11. REFERENCES

[1] T. N. Grossman, L. Roglin, O. Seitz, Angew. Chem. Int. Ed. 2007, 46, 5223-5225; Angew. Chem. 2007, 119, 5315-5318.
[2] L. Osborn, S. Kunkel, G. J. Nabel, Proc. Natl. Acad. Sci. 2006, 86, 2336-2340.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A Beacon-mediated Exponential Amplification Reaction (BEAR) system, comprising;
a polynucleotide beacon, comprising:
a polynucleotide hairpin (HP), comprising: a first overhang region comprising a fluorophore quencher conjugated to the 5′end of said first overhang region, a first stem region positioned 3′ to the first overhang region, a loop region positioned 3′ to the first stem region, a second stem region positioned 3′ to the loop region, such second stem region complementary to the first stem region, and a second overhang region positioned 3′ to the second stem region;
a fluorophore-conjugated polynucleotide (FS), comprising a fluorophore moiety conjugated to the 3′end of said polynucleotide, said fluorophore-conjugated polynucleotide is adapted to reversibly bind to said first overhang region of said polynucleotide hairpin (HP), and said first stem region and loop region of said polynucleotide hairpin (HP),
a polynucleotide primer configured to reversibly bind to a primer binding region that overlaps with said second stem region and said second overhang region of said polynucleotide hairpin (HP) when a target polynucleotide in a sample reversibly binds to said loop region of said polynucleotide hairpin (HP), or when an unbound fluorophore-conjugated polynucleotide (FS) reversibly binds to said first stem region and said loop region of said polynucleotide hairpin (HP); and
a polypeptide with DNA polymerase activity.

2. The Beacon-mediated Exponential Amplification Reaction (BEAR) system of claim 1, wherein each said first overhang region, said first stem region, said loop region, said second stem region, or said primer binding region comprises one or more nucleotide domains.

3. The Beacon-mediated Exponential Amplification Reaction (BEAR) system of claim 1, wherein the concentration of the polynucleotide hairpin (HP) is in excess of the concentration of the fluorophore-conjugated polynucleotide (FS).

4. The Beacon-mediated Exponential Amplification Reaction (BEAR) system of claim 1, wherein said first stem region or second stem region comprises or consists of 14 nucleotides.

5. The Beacon-mediated Exponential Amplification Reaction (BEAR) system of claim 1, wherein said first stem region or second stem region comprises or consists of about 57% content of G and C nucleotide.

6. The Beacon-mediated Exponential Amplification Reaction (BEAR) system of claim 1, wherein the polynucleotide primer comprises or consist of 8 to 11 nucleotides.

7. The Beacon-mediated Exponential Amplification Reaction (BEAR) system of claim 1, wherein said fluorophore moiety is FAM.

8. The Beacon-mediated Exponential Amplification Reaction (BEAR) system of claim 1, wherein said quencher is IOWA BLACK™ FQ.

9. A method of detecting a target polynucleotide in a sample, comprising: contacting a sample with a Beacon-mediated Exponential Amplification Reaction (BEAR) system, said system comprising;
a polynucleotide beacon, comprising:
a polynucleotide hairpin (HP), comprising: a first overhang region comprising a fluorophore quencher conjugated to the 5′end of said first overhang region, a first stem region positioned 3′ to the first overhang region, a loop region positioned 3′ to the first stem region, a second stem region positioned 3′ to the loop region, such second stem region complementary to the first stem region, and a second overhang region positioned 3′ to the second stem region;
a fluorophore-conjugated polynucleotide (FS), comprising a fluorophore moiety conjugated to the 3′end of said polynucleotide, said fluorophore-conjugated polynucleotide is adapted to reversibly bind to said first overhang region of said polynucleotide hairpin (HP), and said first stem region and loop region of said polynucleotide hairpin (HP),
a polynucleotide primer configured to reversibly bind to a primer binding region that overlaps with said second stem region and said second overhang region of said polynucleotide hairpin (HP) when a target polynucleotide in a sample reversibly binds to said loop region of said polynucleotide hairpin (HP), or when an unbound fluorophore-conjugated polynucleotide (FS) reversibly binds to said first stem region and said loop region of said polynucleotide hairpin (HP); and
a polypeptide with DNA polymerase activity.

10. The Beacon-mediated Exponential Amplification Reaction (BEAR) system of claim 1, wherein each said first overhang region, said first stem region, said loop region, said second stem region, or said primer binding region comprises one or more nucleotide domains.

11. The Beacon-mediated Exponential Amplification Reaction (BEAR) system of claim 1, wherein the concentration of the polynucleotide hairpin (HP) is in excess of the concentration of the fluorophore-conjugated polynucleotide (FS).

12. The Beacon-mediated Exponential Amplification Reaction (BEAR) system of claim 1, wherein said first stem region or second stem region comprises or consists of 14 nucleotides.

13. The Beacon-mediated Exponential Amplification Reaction (BEAR) system of claim 1, wherein said first stem region or second stem region comprises or consists of about 57% content of G and C nucleotide.

14. The Beacon-mediated Exponential Amplification Reaction (BEAR) system of claim 1, wherein the polynucleotide primer comprises or consist of 8 to 11 nucleotides.

15. The Beacon-mediated Exponential Amplification Reaction (BEAR) system of claim 1, wherein said fluorophore moiety is FAM.

16. The Beacon-mediated Exponential Amplification Reaction (BEAR) system of claim 1, wherein said quencher is IOWA BLACK™ FQ.

17. A kit, comprising: a polynucleotide beacon, comprising:
a polynucleotide beacon, comprising:
a polynucleotide hairpin (HP), comprising: a first overhang region comprising a fluorophore quencher conjugated to the 5′end of said first overhang region, a first stem region positioned 3′ to the first overhang region, a loop region positioned 3′ to the first stem region, a second stem region positioned 3′ to the loop region, such second stem region complementary to the first stem region, and a second overhang region positioned 3′ to the second stem region;
a fluorophore-conjugated polynucleotide (FS), comprising a fluorophore moiety conjugated to the 3′end of said polynucleotide, said fluorophore-conjugated polynucleotide is adapted to reversibly bind to said first overhang region of said polynucleotide hairpin (HP), and said first stem region and loop region of said polynucleotide hairpin (HP),
a polynucleotide primer configured to reversibly bind to a primer binding region that overlaps with said second stem region and said second overhang region of said polynucleotide hairpin (HP) when a target polynucleotide in a sample reversibly binds to said loop region of said polynucleotide hairpin (HP), or when an unbound fluorophore-conjugated polynucleotide (FS) reversibly binds to said first stem region and said loop region of said polynucleotide hairpin (HP); and
a polypeptide with DNA polymerase activity.

18. The kit of claim 14, wherein the polynucleotide primer comprises or consist of 8 to 11 nucleotides.

19. The kit of claim 14, wherein said fluorophore moiety is FAM.

20. The kit of claim 14, wherein said quencher is IOWA BLACK™ FQ.


SEQUENCE LISTING (12 sequences)

SEQ ID NO: 1 - DNA, 28 bp, synthetic construct
  source 1..28: mol_type = other DNA; note = MERRF Target
gttaaagattaagagagccaacaccaaa

SEQ ID NO: 2 - DNA, 28 bp, synthetic construct
  source 1..28: mol_type = other DNA; note = Negative Control
gttaaagattaagagaaccaacaccaaa

SEQ ID NO: 3 - DNA, 87 bp, synthetic construct
  modified_base 1: mod_base = OTHER; note = Iowa Black FQ
  modified_base 1: mod_base = OTHER; note = Dark Quencher
  source 1..87: mol_type = other DNA; note = HP
tgtgtgtcgtgcgcgttaaagggtgttggctctcggacgcgttaaagggtgttggctctcttaatctttaacgcgtccgtgactttt

SEQ ID NO: 4 - DNA, 33 bp, synthetic construct
  modified_base 33: mod_base = OTHER; note = FAM2
  source 1..33: mol_type = other DNA; note = FS
agagccaacaccctttaacgcgcacgacacaca

SEQ ID NO: 5 - DNA, 11 bp, synthetic construct
  source 1..11: mol_type = other DNA; note = Primer 11
gtcacggacgc

SEQ ID NO: 6 - DNA, 10 bp, synthetic construct
  source 1..10: mol_type = other DNA; note = Primer 5-10
tcacggacgc

SEQ ID NO: 7 - DNA, 10 bp, synthetic construct
  source 1..10: mol_type = other DNA; note = Primer 3-10
gtcacggacg

SEQ ID NO: 8 - DNA, 28 bp, synthetic construct
  source 1..28: mol_type = other DNA; note = MERRF Target
gttaaagattaagagagccaacaccaaa

SEQ ID NO: 9 - DNA, 28 bp, synthetic construct
  source 1..28: mol_type = other DNA; note = Negative Control
gttaaagattaagagaaccaacaccaaa

SEQ ID NO: 10 - DNA, 28 bp, synthetic construct
  source 1..28: mol_type = other DNA; note = Mismatch 1
gttaaagatgaagagagccaacaccaaa

SEQ ID NO: 11 - DNA, 28 bp, synthetic construct
  source 1..28: mol_type = other DNA; note = Mismatch 2
gttgaagattaagagagccaacaccaaa

SEQ ID NO: 12 - DNA, 28 bp, synthetic construct
  source 1..28: mol_type = other DNA; note = Mismatch 3
gttaaagattaagagagccaagaccaaa